(19) United States
(12) Patent Application Publication
(10) Pub. No.: US 2021/0348111 A1
(43) Pub. Date: Nov. 11, 2021

(54) DEVICE FOR INCUBATING CULTURES, METHOD FOR INCUBATING CULTURES AND USE

(71) Applicant: Cellbox Solutions GmbH, Luebeck (DE)

(72) Inventors: Hans Nagels, Bovenden (DE); Christoph Lederer, Berlin (DE)

(73) Assignee: Cellbox Solutions GmbH, Luebeck (DE)

(21) Appl. No.: 17/273,854

(22) PCT Filed: Sep. 6, 2019
(86) PCT No.: PCT/DE2019/000234

§ 371 Date: Mar. 5, 2021

(30) Foreign Application Priority Data
Sep. 6, 2018 (DE) 10 2018 121 781.1

(51) Int. Cl. C12M 1/34; C12M 1/00; C12M 1/107; C12M 3/00; C12M 1/04; C12M 1/36

(52) U.S. Cl. CPC C12M 41/34; C12M 23/34; C12M 23/36; C12M 23/52; C12M 41/48; C12M 27/04; C12M 29/20; C12M 41/40; C12M 23/54

(57) ABSTRACT

The invention relates to a device for incubating cultures, comprising a culture chamber storing the cultures, the cultures being fumigated with an incubation gas. The invention is characterized in that the incubation gas source is used when incubating the cultures with the incubation gas source which is free of liquid incubation gas.

The invention relates to a device for incubating cultures, a method for incubating cultures, and a use relating thereto.

In devices for incubating cultures, the cultures are usually incubated exposed to gas using gaseous CO₂ coming directly from corresponding CO₂ pressure cartridges, wherein this CO₂ is existent in a self-liquefied state and highly pressurized.

Even with precisely working pressure reducing fittings, an extremely precise and, above all, safe incubation, in particular by means of CO₂ in corresponding culture chambers, is not always satisfactorily possible, wherein this applies in particular to portable incubators during transport.

DE 20 2005 021 050 U1 relates to an autonomous incubator for biological material for creating and maintaining a predeterminable CO₂ environment in at least one inner chamber of the incubator including a control device for controlling the CO₂ concentration in the at least one inner chamber, wherein the autonomous incubator comprises a CO₂ incubator.

The problem of the present invention is therefore to provide a device for incubating cultures with which a, with regard to the pressure conditions, precise and reliable metering of the respective incubation gas into the culture chambers is achieved and possible.

According to the invention, this problem is solved by a device according to claim 1, a method according to claim 7, and a use according to claim 8.

The device for incubating cultures according to the invention is one of those comprising a culture chamber storing the cultures, wherein the cultures are aerated with an incubation gas, for example and in particular with CO₂, wherein in use the incubation gas source is free of liquid incubation gas when the cultures are incubated with the incubation gas. With respect to CO₂, this means that, as is usually the case with liquefied CO₂ contained in a pressure cartridge, this is not fed directly into the culture chamber via a pressure reducing fitting and a corresponding valve if required.

Also essential to the invention is the fact that, during the incubation of the cultures with the incubation gas, according to the invention liquid/liquefied incubation gas is dispensed with, wherein the incubation gas CO₂ is in use provided for incubation by sublimation by means of dry ice.

By this embodiment according to the invention, the pressure problems known from the prior art of the expansion of liquid CO₂ to gaseous CO₂ with corresponding expansion of the liquid CO₂ do not arise.

In this context it is further within the meaning of the invention that the incubation gas is CO₂ and in use

a) present as dry ice in a storage container that is open to the outside—i.e. to the ambient atmosphere—for example and in particular in the form of a styrofoam container, or in a styrofoam container that is temporarily open by means of a valve, for example and in particular in the form of a closed styrofoam container designed with a pressure relief valve, wherein the pressure relief valve opens when a certain pressure is exceeded, and then closes again,

since no problematic overpressure conditions arise in the storage container and, for example and in particular by means of a pump, the CO₂ required for the incubation can be charged into the culture chamber.

In this context, it is furthermore advantageous, since tried and tested in practice, that the incubation gas supply is interrupted or enabled upon exceeding or falling below predetermined incubation gas limit values by means of a sensor detecting the incubation gas content of the atmosphere of the culture chamber, for example and in particular, if the preventing or enabling is carried out by means of a valve and/or a metering pump.

Furthermore, it is advantageous that, in the case of CO₂ as the incubation gas, the valve is opened and/or the metering pump is activated for suppling gaseous CO₂ into the culture chamber if the percentage by volume CO₂ in the atmosphere of the culture chamber falls below a predetermined value, for example and in particular 5% by volume CO₂, so that the incubation conditions in the culture chamber are very good.

In addition, it is advantageous if the culture chamber includes a fan for gas exchange in the culture chamber in order to provide a maximally homogeneous composition in respect of the possible different gaseous constituents of the atmosphere in the culture chamber.

Finally, it is within the meaning of the invention that the device according to the invention is designed as a transport container, for example and in particular as a case-like container, in order to safely and under incubation transport sensitive cultures even over long distances, for example and in particular by means of aircraft, even across continents, without the cultures to be incubated and transported being damaged during transport.

In this context, it is advantageous, since tried and tested, if at least one element/part of the device is connected to an inner side of the transport container in an impact-resilient manner.

In this context, it is particularly advantageous, since tried and tested, if the impact-resilient connection is implemented by means of at least one member of the group consisting of an elastic element, for example and in particular a thermoplastic elastomer, for example and in particular polyisobutadiene, a cardanic mounting, and a spring element.

Furthermore, it is advantageous in this context, since tried and tested, if the element/part of the device is one of the group consisting of an incubator, the culture chamber, the storage container, a metering pump, a fan, a cell culture carrier, and a cell culture container.

In addition, it is also advantageous in this context, since tried and tested, if the cell culture carrier is mounted in the culture chamber by means of ball bearings.

Finally, it is advantageous, since tried and tested, if the storage container includes a CO₂ discharge channel to the outside, this being in particular designed as a hose, in order to allow excess gaseous CO₂ to escape in this way.

In this context, it is particularly advantageous, since tried and tested, if the CO₂ discharge channel is disposed on an upper third of the storage container in order to discharge only excess gaseous CO₂ from the storage container and the device.

Finally, it is also within the meaning of the invention if a related method for incubating cultures and/or for transporting cultures is provided, in which a device according to the invention is used.

The same applies to a use according to the invention of the device according to the invention for incubating cultures and/or for transporting cultures.

The invention is in the following explained in detail with the help of an exemplary embodiment that does not restrict the invention, in which

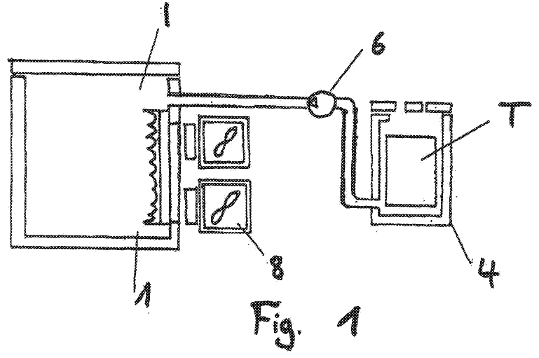

FIG. 1 sketchily shows an embodiment according to the invention;

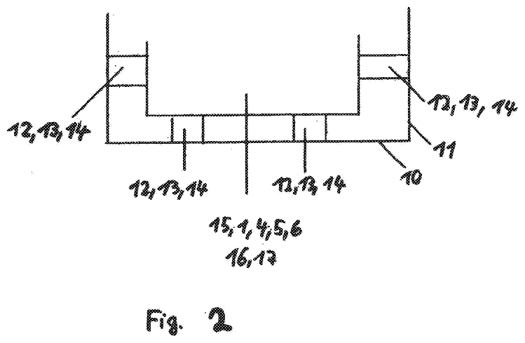

FIG. 2 sketchily shows several embodiments;

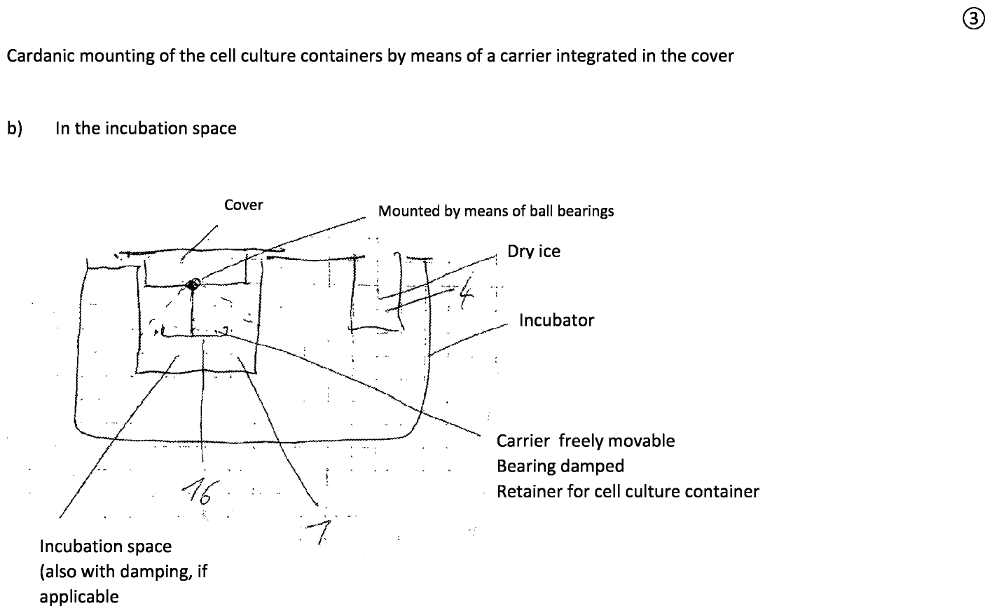

FIG. 3 sketchily shows another embodiment according to the invention;

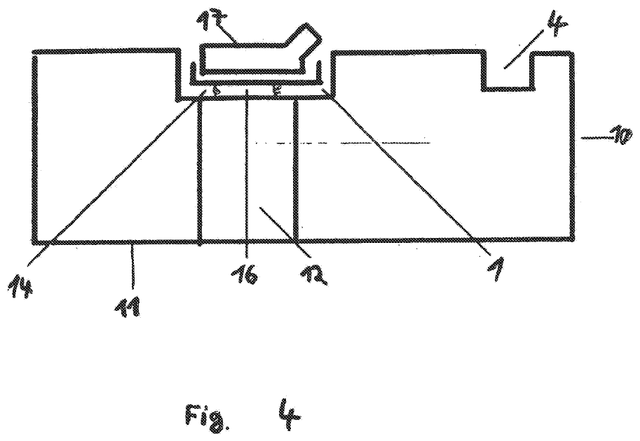

FIG. 4 sketchily shows a third embodiment according to the invention;

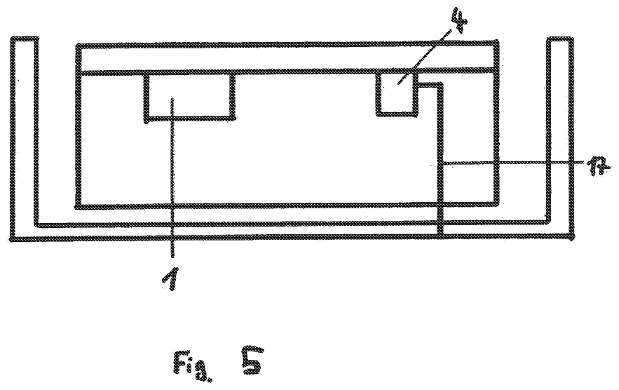

FIG. 5 sketchily shows another embodiment according to the invention.

The embodiment of FIG. 1 shows that the storage container 4 is a container that is open to the ambient atmosphere, for example and in particular a styrofoam container containing one or more pieces of dry ice T, so that CO₂ passes continuously and controlled into the gaseous phase by sublimation and, if necessary, can be charged into the atmosphere of the culture chamber 1 by means of a valve (not shown) and/or, for example and in particular, a metering pump 6, wherein the culture chamber 1 includes, for example and in particular, one or more fans 8 for swirling, and these thus contribute to the homogenization of the atmosphere of the culture chamber 1.

FIG. 2 shows the basic principle of the manner in which elements of the device are connected to an inner side 11 of the transport container 10 in an impact-resilient manner. The incubator 15, the culture chamber 1, the storage container 4 for the CO₂, the metering pump 6, the fan 5, as well as the cell culture carrier 16 and the cell culture container 17 can be mounted by impact-resilient connection via elastic elements 12, cardanic mountings 13, and spring elements 14 on the inner side 11 of the transport container 10.

According to the invention, a cardanic mounting is to be understood as the suspension of at least one above-mentioned element, for example and in particular, from an inner side 11 of the transport container 10, for example and in particular with the aid of two intersecting pivot bearings that are arranged perpendicular to each other.

FIG. 3 shows a sketch of a cell culture carrier 16 (not shown in detail) being mounted by ball bearings in a culture chamber 1—next to a storage container 4 in which dry ice is stored.

FIG. 4 shows a sketch of a cell culture container 17 which abuts on a cell culture carrier 16, wherein the cell culture carrier 16 abuts on spring elements 14 within a culture chamber 1, the culture chamber 1 itself being also connected to an inner side 11 of the transport container 10 via an elastic element 12.

A further embodiment of the device according to the invention is sketchily shown in FIG. 5, where in a storage container 4, in which dry ice is stored during use, a CO₂ discharge channel 17 in the shape of a hose is disposed on an upper third in order to all but automatically discharge excess CO₂ out of the device according to the invention, wherein the required CO₂ can be fed into a culture chamber 1 for the incubation, for example, by means of a valve (not shown in detail).

1. Device for incubating cultures, comprising:
a culture chamber storing the cultures,
a storage container which is open to an outside or comprises a valve configured to temporarily open the storage container to the outside and which is configured to receive dry ice,
wherein, during use:
the cultures are aerated with an incubation gas an incubation gas source is free of liquid incubation gas when the cultures are incubated with the incubation gas, the incubation gas is provided for incubation via sublimation, wherein the incubation gas is CO₂ and is provided as the dry ice in the storage container and, wherein the device is designed as a transport container.

2. The device according to claim 1, further comprising a sensor, wherein the device, via the sensor, is configured: to detect the incubation gas content of an atmosphere of the culture chamber and to interrupt or enable a supply of incubation gas upon exceeding or falling below predetermined incubation gas limit values.

3. The device according to claim 2, further comprising a valve and/or a metering pump, wherein the device is configured to carry out the interrupting or enabling of the supply of the incubation gas via the valve and/or the metering pump.

4. The device according to claim 3, wherein, if a percentage by volume CO₂ in the atmosphere of the culture chamber falls below a predetermined value, the valve is opened and/or the metering pump is activated for suppling gaseous CO₂ into the culture chamber.

5. The device according to claim 1, wherein the culture chamber further comprises a fan for gas exchange in the culture chamber.

6. The device according to claim 1, wherein the transport container is designed as a case-like container.

7. The device according to claim 6, wherein at least one element of the device is connected to an inner side of the transport container in an impact-resilient manner.

8. The device according to claim 7, wherein the impact-resilient connection is implemented via at least one member of the group consisting of an elastic element, a cardanic mounting, and a spring element.

9. The device according to claim 7, wherein the element of the device is one of the group consisting of an incubator, the culture chamber, the storage container, a metering pump, a fan, a cell culture carrier, and a cell culture container.

10. The device according to claim 9, wherein the cell culture carrier is mounted in the culture chamber via ball bearings.

11. The device according to claim 1, wherein the storage container comprises a CO₂ discharge channel to the outside.

12. The device according to claim 11, wherein the CO₂ discharge channel is designed as a hose.

13. The device according to claim 11, wherein the CO₂ discharge channel is situated at an upper third of the storage container.

14. Method for incubating cultures and/or for transporting cultures, comprising:
providing a device according to claim 1, wherein, during use, the cultures are aerated with CO₂ and the CO₂ is provided via sublimation of the dry ice.

15. The method according to claim 14, wherein the use is incubating the cultures and/or transporting the cultures in the culture chamber.

16. The method according to claim 15, wherein the use is incubating the cultures and transporting the cultures.

17. The method according to claim 14, wherein, if a percentage by volume CO₂ in an atmosphere of the culture chamber falls below a predetermined value, a valve is opened and/or the metering pump is activated for suppling gaseous CO₂ from the storage container into the culture chamber.
